(19) United States
(12) Patent Application Publication
(10) Pub. No.: US 2024/0382748 A1
(43) Pub. Date: Nov. 21, 2024

(54) Pacing lead with advancing stylet and methods of implantation thereof

(71) Applicant: Eagle Point Medical LLC, City of Dover, DE (US)

(72) Inventor: Leonid Makharinsky, Bonassola (IT)

(21) Appl. No.: 18/406,168

(22) Filed: Jan. 7, 2024

Related U.S. Application Data
(60) Provisional application No. 63/467,058, filed on May 17, 2023.
(60) Provisional application No. 63/471,479, filed on Jun. 6, 2023.

(51) Int. Cl. A61N 1/05; A61N 1/37

(52) U.S. Cl. CPC A61N 1/0565; A61N 1/3702

(57) ABSTRACT

A cardiac pacing lead comprises an elongated flexible body with one or more lead electrodes positioned at a distal end thereof. The lead electrode may be electrically connected to one or more lead connectors at the proximal end, which is configured to transmit electrical signals to or from the distal lead electrode. The elongated flexible body may include a lumen in the center of the lead cross-section. The lumen may be sized to slidingly accept an electrically active elongated stylet positioned therein. The elongated stylet comprises one or more stylet electrodes located at a distal end thereof and electrically connected to one or more stylet connectors at a proximal end thereof. These connectors may be used to transmit electrical signals to or from corresponding distal stylet electrodes. The implantation method includes a step of position pre-checking using the elongated stylet before committing to implantation of the pacing lead.

### CROSS-REFERENCE DATA

This patent application claims a priority date benefit from the U.S. Provisional Patent Application No. 63/471,479 filed 6 Jun. 2023 by the same inventor and entitled “SYSTEMS AND METHODS FOR POSITION PRE-CHECK AND ATRAUMATIC CARDIAC PACING LEAD IMPLANTATION,” incorporated herein by reference in its entirety.

This patent application further claims a priority date benefit from the U.S. Provisional Patent Application No. 63/467,058 filed 17 May 2023 by the same inventor and entitled “LEFT BUNDLE BRANCH PACING CATHETER WITH DEPTH CONTROL USING A MULTI-ELECTRODE LEAD WITH A FIXED OR AN EXTENDABLE-RETRACTABLE TIP,” also incorporated herein by reference in its entirety.

### BACKGROUND

Without limiting the scope of the invention, its background is described in connection with pacing leads, in particular cardiac pacing leads. More particularly, the invention is concerned with cardiac pacing leads designed for implantation into cardiac tissue, such as an interventricular septum, for treating various heart conditions associated with cardiac arrhythmias and symptomatic bradycardias, as well as conditions associated with the left bundle branch (LBB) block or similar conduction disturbances associated with cardiac dyssynchrony and subject to cardiac resynchronization therapy.

Implanting a cardiac pacing lead into the interventricular septum presents several challenges. First, the procedure requires precise navigation through the heart's complex and dynamic structure, where the risk of damaging critical cardiac tissue is significant. The interventricular septum, being a muscular wall dividing the left and right ventricles, poses a challenge in terms of accessing and securing the lead in the correct position.

Accurate placement is crucial, as improper positioning can lead to inadequate pacing or complications like cardiac perforation. Additionally, there's a risk of lead dislodgement at a later point after the implantation procedure, which can cause the device to malfunction. This risk is heightened by the septum's constant motion and the pressure differences between the ventricles during the cardiac cycle.

Moreover, patients with complex cardiac anatomies, such as those with congenital heart defects or significant cardiac remodeling due to diseases like hypertrophic cardiomyopathy, present additional challenges. In these cases, the standard approach may need to be modified, demanding a higher level of expertise and customization of the procedure.

Once the pacing lead is implanted at the first position in the septum that appears to be suitable, its function is checked to ensure proper delivery of cardiac pacing. If such pacing is not adequate, the lead needs to be repositioned and implanted again in another location at the cardiac tissue nearby.

The lead implantation procedure has a number of risks and a potential for significant tissue damage. Multiple lead repositioning deep into the septum which may be required for the new direction in cardiac pacing—conduction system pacing—may result in multiple “drilled” holes into the muscular structure (myocardial damage, etc.). Pacing lead dislodgement, which may result from insufficient tip fixation or depth, can lead to inadequate pacing and necessitate additional procedures for correction. Repositioning can also increase the danger of perforating heart tissue, posing a severe risk of complications like cardiac tamponade. The procedure's duration is extended with each repositioning attempt, exposing the patient to heightened risks such as infection, increased anesthesia effects, and greater radiation exposure from prolonged fluoroscopy. Moreover, extensive maneuvering with the lead through the venous system can traumatize the veins, potentially leading to thrombosis or stenosis.

Tissue damage is another significant concern. The process of implanting and adjusting the lead can cause myocardial trauma, leading to inflammation, edema, or even necrosis. There's also a risk of inducing additional arrhythmias due to manipulation of sensitive areas in the heart's conduction system. In right heart procedures, there's a possibility of damaging the tricuspid valve, resulting in valve regurgitation and hemodynamic compromise. The lead tip, if improperly positioned, can cause focal injury and scarring, affecting the electrical impulse transmission. Lastly, the body's natural inflammatory response to the foreign object of the pacing lead can lead to fibrous tissue formation around the lead, complicating future repositioning or removal.

These challenges highlight the need for a novel pacing lead and novel lead implantation methods that minimize the need for repositioning and reduce overall tissue trauma during the procedure.

### SUMMARY

Accordingly, it is an object of the present invention to overcome these and other drawbacks of the prior art by providing a novel cardiac pacing lead configured to allow a pre-check of the implantation position before fully implanting the lead into cardiac tissue.

It is another object of the present invention to provide a novel cardiac pacing lead configured to reduce the extent of tissue damage during lead implantation procedure.

It is a further object of the present invention to provide a novel pacing lead configured to reduce the time required for pacing lead implantation.

It is yet a further object of the present invention to provide a method for implantation of a cardiac pacing lead aimed at minimizing the risks to the patient and reduce cardiac tissue trauma during the procedure.

The cardiac pacing lead of the present invention may include an elongated flexible body with at least one lead electrode positioned at a distal end thereof. In other embodiments, the distal end of the elongated body may include two, three, or more electrodes, as well as a defibrillation coil or other electrically active components known in the art. The lead electrode may be electrically connected to at least one lead connector at the proximal end of the elongated body. The elongated body and its components may be configured to transmit electrical signals to or from the distal lead electrode using the proximal lead connector.

The elongated body may include a lumen, such as located in the center of the lead cross-section. The lumen may be sized to slidingly and, optionally, removably accept an electrically active elongated stylet positioned therein. The elongated stylet may feature at least one stylet electrode located at a distal end thereof. In other embodiments, more than one electrode may be positioned at the distal end of the elongated stylet and configured to be electrically connected to at least one or more stylet connectors at a proximal end thereof. These connectors may be used to transmit electrical signals to or from corresponding distal stylet electrodes.

A novel method of implanting a cardiac pacing lead and providing a pacing therapy to a target cardiac tissue may include the steps of:
a. providing the cardiac pacing lead with a lead electrode at a distal end thereof and a lumen sized to slidingly and removably accept an elongated stylet positioned therein. The elongated stylet may include at least one stylet electrode located at a distal end thereof. The stylet electrode may be configured to be electrically connected to a stylet connector at a proximal end thereof. Overall, the elongated stylet may be configured to transmit electrical signals to or from the stylet electrode from the proximal end thereof;
b. positioning a distal end of the pacing lead adjacent to a target cardiac tissue, with the elongated stylet preloaded in the lumen of the pacing lead or inserted once the pacing lead is in position;
C. advancing the elongated stylet through and out of the lumen in the cardiac pacing lead to position the stylet electrode at a first position for implantation of the cardiac pacing lead;
d. pre-check the first position for implantation in the cardiac tissue by temporarily confirming the ability to deliver the pacing therapy thereat using the stylet electrode, such as by temporary pacing and capturing the target cardiac tissue and/or by recording an electrogram indicating appropriate target;
e. if the confirmation is not reached in step (d), changing the depth of the stylet electrode of the elongated stylet by gradually inserting it further into the cardiac tissue. This can be repeated more than once until the depth limit indicated by other methods familiar to those skilled in the art, is reached. If that does not produce the desired result at the first position, repositioning the distal end of the cardiac pacing lead and the elongated stylet to a second or further position at the cardiac tissue and repeating step (d) of temporarily confirming the ability to deliver the pacing therapy at the current position and depth of the stylet electrode;
f. upon reaching the confirmation of the position being adequate for delivery of pacing therapy in step (d) or step (e), deploying the cardiac pacing lead to locate the lead electrode next to the stylet electrode of the elongated stylet. This can be done by rotating the pacing lead and advancing the helical tip along the elongated stylet toward the stylet electrode; and
g. withdrawing the elongated stylet while leaving the cardiac pacing lead in the target cardiac tissue.

In embodiments, a step of improving orthogonality of implantation may be performed after step (b) which may include a provisional shallow engagement of the pacing lead with the cardiac tissue to anchor the pacing lead at the surface of the cardiac tissue. The proximal end of the pacing lead may be pushed, pulled, or rotated to cause the distal end to be oriented perpendicularly to the surface of the cardiac tissue. Step (c) may then be attempted to cause the distal end of the elongated stylet to be implanted into cardiac tissue at the desired orthogonal orientation thereto.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a schematic side cross-sectional view of the novel pacing lead of the invention,

FIGS. 2a through 2d show basic method steps of the present invention,

FIGS. 3a through 3f show steps to improve the orthogonality of lead implantation,

FIG. 4 shows a perspective view of one embodiment of the distal end of the pacing lead, according to the invention,

FIG. 5 shows a side cross-sectional view of the same,

FIG. 6 shows a perspective view of the same with the elongated stylet positioned therein,

FIG. 7 shows a side view of the distal end of the pacing lead according to another embodiment of the invention,

FIG. 8 shows a side view of the distal end of the pacing lead according to yet another embodiment of the invention,

FIG. 9 shows a side view of the distal end of the pacing lead according to a further embodiment of the invention,

FIG. 10 shows a side view of the distal end of the pacing lead according to yet a further embodiment of the invention,

FIG. 11 shows a side view of the distal end of the pacing lead according to yet another embodiment of the invention,

FIG. 12 shows a side cross-sectional view of the distal end of the pacing lead having an extendable helical tip at the distal end, according to another embodiment of the invention,

FIG. 13 shows a side cross-sectional view of the distal end of the pacing lead having an extendable helical tip at the distal end, according to yet another embodiment of the invention,

FIG. 14 shows a side cross-sectional view of the distal end of the pacing lead having an extendable helical tip at the distal end, according to a further embodiment of the invention,

FIG. 15 shows a schematical side view of the proximal end of the pacing lead,

FIG. 16 shows a close-up side view of a connector at the proximal end of the pacing lead,

FIG. 17 shows a proximal end of the elongated stylet,

FIG. 18 shows the proximal end of the stylet assembled into a transition hub,

FIG. 19 shows a side view of the assembly of FIG. 18 further attached to a proximal end of the pacing lead,

FIG. 20 shows an alternative configuration of the proximal end of the elongated stylet assembled at the proximal end of the pacing lead,

FIG. 21 shows a first position of an alternative configuration of the transition hub for attaching the proximal end of the elongated stylet to the proximal end of the pacing lead,

FIG. 22 shows a second position of an alternative configuration of the transition hub for attaching the proximal end of the elongated stylet to the proximal end of the pacing lead,

FIG. 23 shows the distal end of the elongated stylet with one stylet electrode, and

FIG. 24 shows the distal end of the elongated stylet with multiple stylet electrodes.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

### Conceptual Design of the Lead and an Electrically Active Elongated Stylet

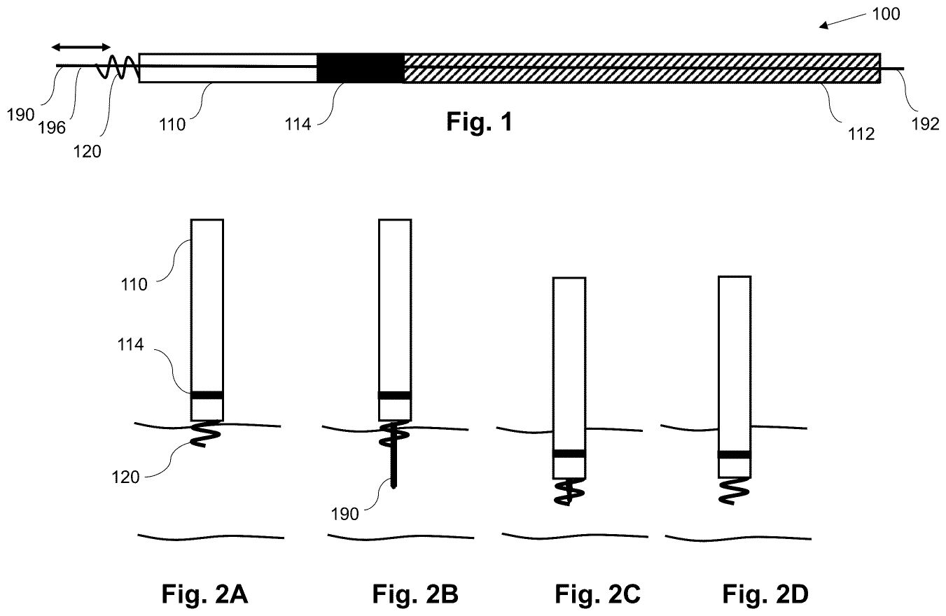

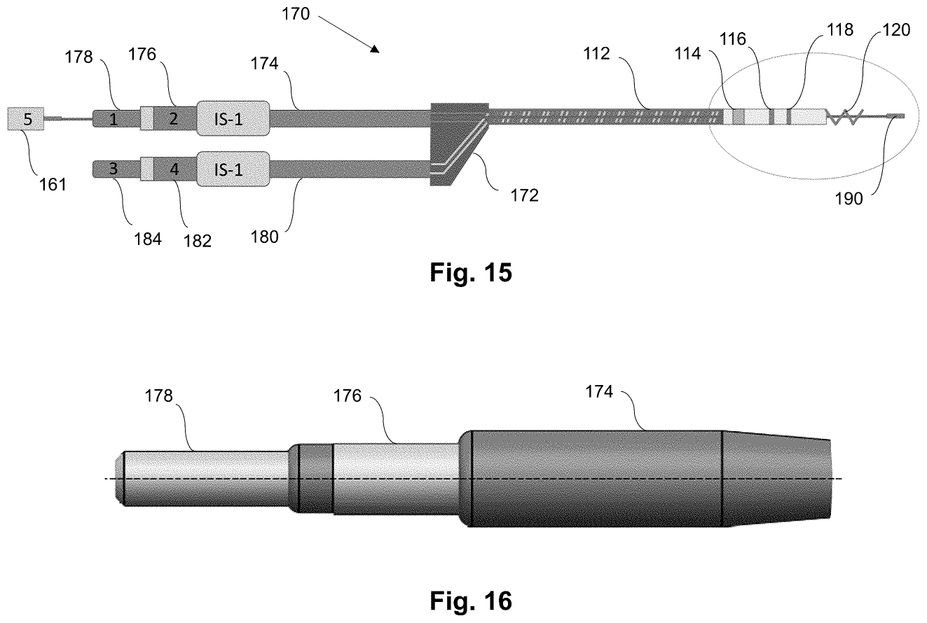

FIG. 1 shows a general cross-sectional view of an exemplary distal end of a pacing lead according to the invention. The pacing lead 100 may include an elongated body 112, which may be made from a biocompatible polymer sleeve. The elongated body 112 may define a distal end 110 and a proximal end 170 (seen in greater detail in FIG. 15). The distal end 110 may have a helical tip 120 for securing the pacing lead in the cardiac tissue, which may be fixed or extendable from the distal end 110, as described in greater detail below. At least one lead electrode may be formed at the distal end of the lead 100, such as at the helical tip 120 or as a ring electrode 114.

Additional lead electrodes may also be formed, as described below, as the invention is not limited in this regard. Such additional electrodes may be used to monitor impedance and, therefore, determine the depth of implantation, as described in greater detail in my other patents, as well as in this paper: Orlov M V, Nikolaychuk M, Koulouridis I, Goldman A, Natan S, Armstrong J, Bhattacharya A, Hicks A, King M, Wylie J. Left bundle area pacing: Guiding implant depth by ring measurements. Heart Rhythm. 2023 Jan.; 20 (1): 55-60. doi: 10.1016/j.hrthm.2022.09.013. PMID: 36152975.

The pacing lead 100 may have an internal lumen extending from the proximal end 170 to the distal end 110 thereof. The lumen may be sized and configured to house an elongated stylet 192 slidably positioned therein. The elongated stylet 192, in turn, may feature at least one stylet electrode 190 at the distal end 196 thereof. The stylet electrode 190 may be configured to electrically communicate with the stylet connector 161 located at the proximal end of the elongated lead 192 to transmit electrical signals. The elongated stylet 192 may be placed in the lumen of the pacing lead 100 with the ability to slide back and forth and protrude from the distal end 110 of the pacing lead 100 beyond the helical tip 120. The length of the elongated stylet may be selected to exceed the length of the pacing lead 100 so as to allow the distal stylet electrode 190 to extend beyond the helical tip 120 while still controlling the stylet 192 at its proximal end protruding back from the corresponding proximal end 170 of the pacing lead 100.

### Method of Implantation of the Pacing Lead

Steps of the novel implantation method are described in connection with FIGS. 2a through 2d. The implantation procedure may start with providing the cardiac pacing lead 100 as described above. The pacing lead 100 may have a helical tip 120 configured to retain the pacing lead in the cardiac tissue when deployed at the suitable position. The helical tip 120 may be configured to act as a lead electrode at the distal end thereof. The pacing lead 100 may have one or more additional distal lead electrodes, such as a first distal lead electrode 114. The pacing lead 100 may have a lumen sized to slidingly and removably accept an elongated stylet 192 positioned therein. The elongated stylet 192 may include at least one stylet electrode 190 located at a distal end thereof. The stylet electrode 190 may be configured to be electrically connected to a stylet connector 161 at a proximal end thereof (shown in FIG. 15). Overall, the elongated stylet 192 may be configured to transmit electrical signals to or from the stylet electrode 190 from the proximal end thereof.

The next step of the method may be a step of positioning the distal end 110 of the pacing lead 100 adjacent to a first target position at the cardiac tissue, with the elongated stylet 192 either preloaded in the lumen of the pacing lead 100 or inserted once the pacing lead 100 is in position. This step may be accomplished using conventional techniques of pacing lead implantation via the venous system of the patient and through the right atria of the heart, as may be known to a person skilled in the art. A suitable sized and shaped delivery sheath may be used for that purpose (not shown in the drawings).

As an optional next step, the helical tip 120 of the pacing lead 100 may be provisionally engaged with the cardiac tissue to anchor the distal end 110 of the pacing lead 100 therein—as seen in FIG. 2a. Such provisional engagement may be done by implanting the helical tip 120 to a shallow depth, such as 1-3 mm, which is sufficient for a temporary anchoring purpose. As explained below, this provisional engagement of the helical tip 120 may be useful for the purposes of improving the orthogonality of lead implantation. In other embodiments of the method, no such provisional engagement may be necessary.

The next step of the method may be a step of advancing the elongated stylet 192 through the lumen in the cardiac pacing lead 100 and out of the distal end 110 thereof to position the stylet electrode 190 at a desirable depth of the first position for implantation—as seen in FIG. 2b.

Once the stylet electrode 190 is in place, a step of pre-checking the first position for implantation in the cardiac tissue may be performed. This may be done by temporarily confirming the ability to deliver the pacing therapy thereat using the stylet electrode 190. In one example, this step may further include a step of pacing the heart using the stylet electrode 190. As an alternative, or in addition, it may further include a step of monitoring an intracardiac electrogram signal. Such signal may be acquired using the stylet electrode 190 or other electrodes configured to record the electrical activity of the heart.

Additional distal electrodes may be provided on one or both distal ends of the pacing lead 100 and the elongated stylet 192. Such additional electrodes may be used during the insertion procedure to monitor impedance or other pacing parameters and therefore determine the depth of implantation for the elongated stylet and/or the pacing lead, as described in greater detail in my other patents. Additional distal electrodes may also be used for image-based depth determination using X-Ray or fluoroscopy, as they have excellent radiopacity.

If the pacing therapy is selected to pace a left bundle branch area, in step (d) or step (e) the step of temporarily confirming the ability to deliver the pacing therapy may include a step of temporary pacing the left bundle branch area using the stylet electrode of the elongated stylet to verify the ability to capture thereof. Furthermore, it can be used for additionally recording a specific electrogram from the same electrode.

The capturing of the conduction system of the heart may be confirmed using one or several criteria as accepted in the field of cardiac pacing. For example, Marek Jastrzębski et al. discuss various criteria for determining the capture of the conduction system, see Jastrzębski M et al. Left bundle branch area pacing outcomes: the multicentre European MELOS study, European Heart Journal (2022) 43, 4161-4173, incorporated herein in its entirety by reference. Other criteria may also be used, as the present invention is not limited in this regard—it provides the necessary tool to temporarily assess and, therefore, pre-check the position of the implantation of a cardiac pacing lead prior to the actual implantation thereof at that location.

Several other criteria may be considered to be indicative of left bundle branch area capture, such as the following non-limiting examples:
i. appearance of a characteristic paced QRS complex on the 12-lead ECG, typically showing an incomplete right bundle branch block, when the lead reaches the final position near the left bundle branch,
ii. short peak left ventricular activation time as defined in a Vijayaraman's publication (Vijayaraman P, Subzposh F A, Naperkowski A, et al. Prospective evaluation of feasibility and electrophysiologic and echocardiographic characteristics of left bundle branch area pacing. Heart Rhythm 2019; 16:1774-1782.), typically less than 80 ms from the stimulus to the peak of the QRS in lead V6 in patients with narrow baseline QRS,
iii. two different paced QRS morphologies during threshold testing may be considered to be indicative of conduction system capture,
iv. a delay of more than 40 ms between the QRS peak in lead V6 and lead V1,
V. characteristic changes in pacing impedance and other pacing parameters indicative of the electrode reaching a certain depth in the septum may also be used to access the lead progression into the septum (see Orlov M V, Nikolaychuk M, Koulouridis I, Goldman A, Natan S, Armstrong J, Bhattacharya A, Hicks A, King M, Wylie J. Left bundle area pacing: Guiding implant depth by ring measurements. Heart Rhythm. 2023 Jan.; 20 (1): 55-60).

Alternatively, or in addition, the step of temporarily confirming the ability to deliver the pacing therapy using the elongated stylet may further include a step of verifying a pacing capture of the cardiac tissue or the presence of a predetermined feature on the cardiac electrogram, as known in the art of cardiac pacing. Recording of certain electrogram characteristics may be considered indicative of the first distal end electrode being in the vicinity of the left bundle branch, such as a recording of a discrete left bundle branch potential, typically 25-35 ms in front of the QRS, or a discrete sharp signal indicative of Purkinje potential recorded less than 25 ms in front of the QRS.

Optionally, the step of pre-checking the position for implantation in the cardiac tissue by temporarily confirming the ability to deliver the pacing therapy thereat may include a step of confirming the pacing location using a predetermined acceptability criterion. Such acceptability criterion may be based on an ECG recording at the target site as described above or on image-based guidance, for example, using fluoroscopy to identify the position of the stylet electrode 190.

Furthermore, if the distal end of the elongated stylet includes a stylet electrode and at least one or more additional distal stylet electrodes, the step of temporarily confirming the ability to deliver the pacing therapy may include a step of temporary pacing and monitoring an intracardiac electrogram using either two or more of any available stylet electrodes—in a unipolar pacing fashion or in a bipolar pacing fashion.

If the intended cardiac pacing therapy is to pace the left bundle branch area, the step of temporarily confirming the ability to deliver the pacing therapy may further include a step of temporarily pacing the left bundle branch area using the stylet electrode to verify the ability to capture thereof.

If the desired confirmation is not reached in the previous step, the depth of the stylet electrode 190 may be gradually changed by inserting it further into the cardiac tissue. The stylet electrode 190 may be continuously or intermittently monitored to detect the depth of the insertion—for example, by using impedance measurements, as described in my previous patent applications. The stylet electrode 190 may be used to record paced ECG, conduction system potentials, and other suitable metrics as may be customary. In addition, monitoring the position of the proximal end of the elongated stylet 192 in relationship to the proximal end 170 of the pacing lead 100 and/or observing the distal end 196 of the elongated stylet may be conducted using fluoroscopy or other imaging methods. In that sense, the elongated stylet 192 may be used in a manner similar to that of a pacing wire. Once the desired depth is reached, a temporary pacing therapy delivery may be verified using the stylet electrode 190. If that still does not produce the desired result, the distal end 110 of the cardiac pacing lead 100 and the elongated stylet 192 may be moved to a second or further position at the cardiac tissue. As can be appreciated by those skilled in the art, inserting the elongated stylet 192 deeper into the cardiac tissue may only be done within acceptable depth limits, as can be determined by other methods, so as to avoid tissue perforation.

Importantly, at this point in the procedure, the only trauma to the cardiac tissue is the penetration of a thin stylet tip 196 and not the much larger helical tip 120 of the cardiac pacing lead 100. The procedure of verifying temporary pacing therapy delivery may be repeated one or more times until the proper implantation position and a suitable implantation depth are found. Although the trauma to the heart tissue is minimal, it is suggested that redeployment of the elongated stylet 192 and the pacing lead 100 is not done more than a few times in order to further limit tissue damage.

Upon reaching the confirmation of the position being adequate for delivery of pacing therapy, the helical tip 120 of the cardiac pacing lead 190 may be advanced over the elongated stylet 192 as a monorail to locate the lead electrode next to the stylet electrode 190—see FIG. 2c. The elongated stylet 192 may then be withdrawn while leaving the cardiac pacing lead 100 in the target cardiac tissue. The empty channel formed in the lumen of the pacing lead 100 may be used in the future for injecting a drug or a contrast solution, or to deliver another form of local therapy, as well as for removal purposes when the pacing lead 100 is no longer functional or no longer needed.

The implantation procedure may be performed using a suitable delivery sheath. One example of a suitable delivery sheath may be a sheath having a preferred curve at the distal end thereof. Another example is a delivery sheath configured to be deflectable to assist in positioning of the distal end of the pacing lead at the target cardiac tissue.

### Improvement of Orthogonality of Lead Implantation

In addition to the steps described above, the present invention may allow for an optional maneuver described below aimed at improving the quality of lead implantation, namely at achieving implantation at about a 90-degree angle to the target cardiac tissue. A desire to do so is described in various publications, for example, in Vijayaraman P, Subzposh F A, Naperkowski A, et al. Prospective evaluation of feasibility and electrophysiologic and echocardiographic characteristics of left bundle branch area pacing. Heart Rhythm 2019; 16:1774-1782.

The approach direction through the tricuspid valve to implant the pacing lead into the interventricular septum, for example, may not necessarily be at an optimal 90-degree angle to the septal wall. Various curved shapes of the distal end of the delivery sheath are typically used to make the approach more orthogonal, but it may still not be able to accomplish the desired orientation of the pacing lead and cardiac tissue. The present invention may also be advantageously used to further improve the pacing lead deployment and achieve an optimal orthogonal orientation of the distal end of the lead during deployment.

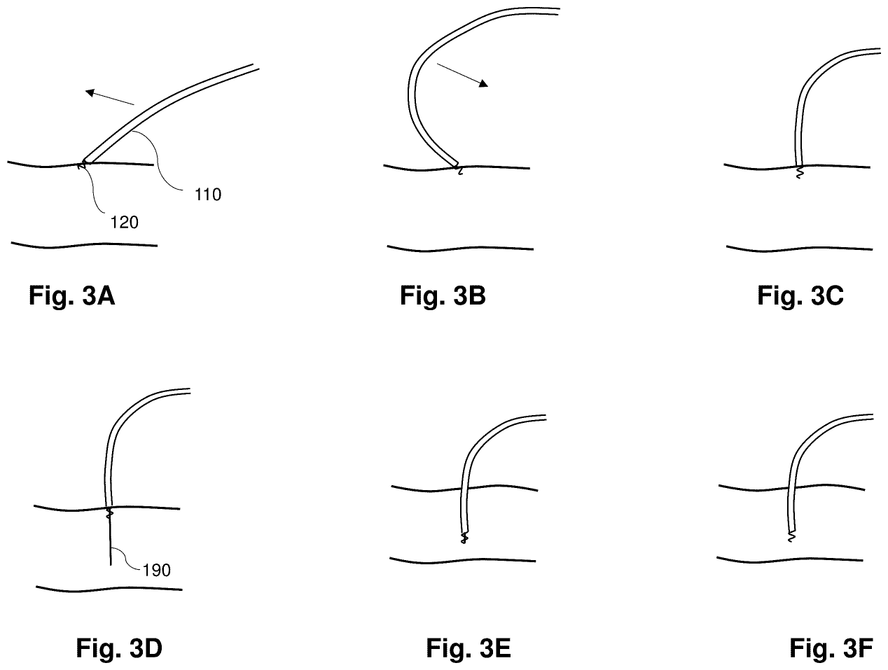

FIGS. 3a through 3f illustrate the additional steps of the method of implantation according to the invention. The initial approach of the distal end of the pacing lead 100 containing the elongated stylet 190 may be either at: (i) an acute angle to the septum, as shown in FIG. 3a, (ii) at an obtuse angle to the septum, as seen in FIG. 3b, or (iii) the plane of the pacing lead curve may itself be at an acute or an obtuse side angle to the septum.

The present invention may be used to facilitate optimal pacing lead delivery in the following way. Once the distal end of the pacing lead 100 is positioned next to the cardiac tissue at the first or at additional positions targeted for implantation, the helical tip 120 may be advanced to provisionally engage with the underlying tissue at a “shallow” depth, typically 1-3 mm or so as described above. The distal end of the pacing lead 110 is now “hooked” onto the cardiac tissue and acts as an anchor and allows the operator to manipulate and pivot the delivery sheath or the pacing lead 100 to achieve a proper angle of implantation for the remaining steps of the procedure.

If the initial angle between the distal end of the pacing lead 100 and the underlying tissue is acute, as seen in FIG. 3a, the operator may push on the delivery sheath and advance it forward, as indicated by the arrow in this drawing. This causes a bowing effect of the pacing lead 100 to arch into a desired position shown in FIGS. 2a and 3c.

If the initial angle between the distal end of the pacing lead 100 and the underlying tissue is obtuse, as seen in FIG. 3b, the operator may pull back on the pacing lead 100 (see arrow in FIG. 3b) to achieve the same proper angle as seen in FIG. 3c.

If the plane of the delivery sheath curve is tilted to the surface of the cardiac tissue forming a side angle therewith, the operator may apply torque in the appropriate direction to rotate the plane to be perpendicular to the cardiac tissue.

As can be understood by a person skilled in the art, a combination of pull and torque or push and torque may be required in order to take advantage of the distal tip of the pacing lead 100 acting as an anchor in the cardiac tissue. Additionally or alternatively, the same maneuvers can be applied to the delivery sheath to facilitate the same goal of orthogonality to the external surface of the cardiac tissue.

Once the orientation of the pacing lead 100 is satisfactory, the operator may proceed to implant the stylet electrode 190 to a desired depth, as seen in FIG. 3d. Position pre-check may be performed as explained in greater detail above and, once confirmed, the pacing lead 100 may be implanted to the same position over the stylet electrode 190—see FIG. 3e. The elongated stylet 192 may then be withdrawn as seen in FIG. 3f, leaving the cardiac pacing lead 100 implanted into cardiac tissue at the optimal orientation and at desired depth.

Pacing Lead with a Fixed Helical Tip

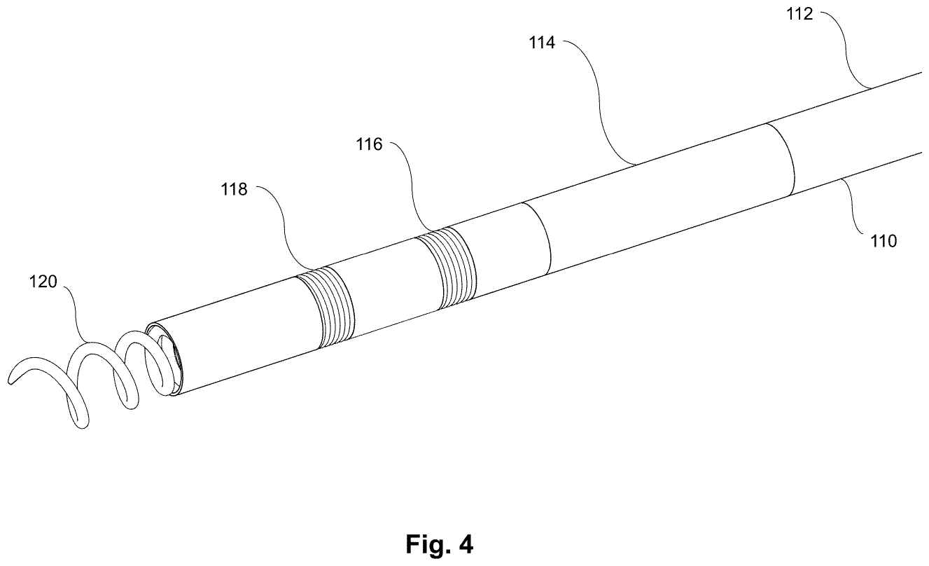

The present invention may be practiced with a variety of pacing leads, including pacing leads with a fixed helical tip as well as pacing leads with an extendable helical tip. FIGS. 4 through 11 illustrate various designs of the novel lead having a fixed helical tip, which are now described in greater detail.

The term “helical tip” is used herein to broadly describe spiral and other suitable tissue fixation tips that are generally known in the art of securing a pacing lead to the target tissue, as the invention is not limited to just a tip having a strictly helical geometry. In embodiments, the geometry of the helical tip may be selected such that it allows the elongated stylet to pass through the outer boundaries of the tip, such as to pass along its central longitudinal axis. The central opening or open space at the center of the helical tip may be selected to be at least that of the external diameter of the elongated stylet or larger, depending on the design preferences and specific lead considerations.

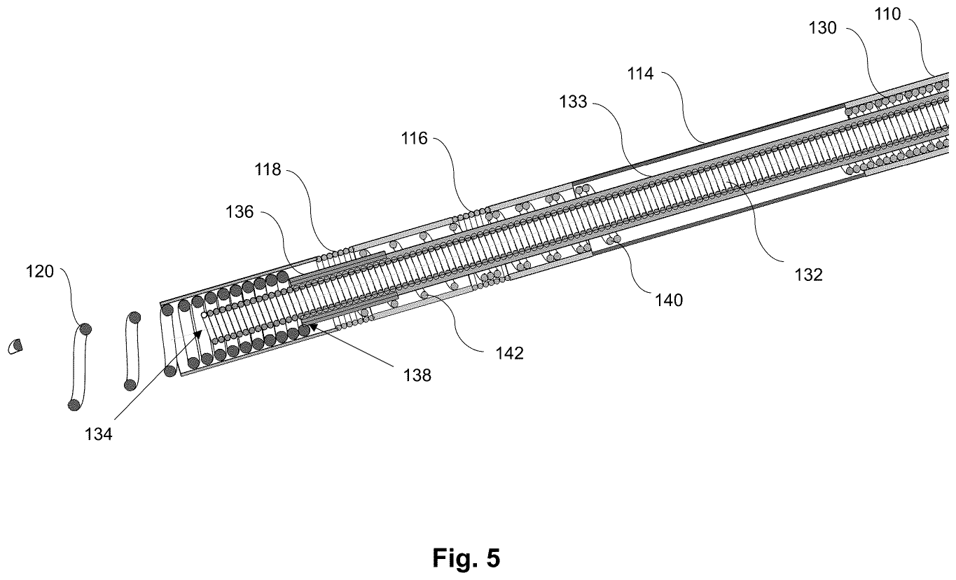

A general perspective view of a distal and of a suitable pacing lead with a fixed helical tip at the distal end is seen in FIG. 4, with a cross-sectional view shown in FIG. 5. The pacing lead 100 contains an elongated body 112 with the distal end 110. The elongated body 112 may be made from a suitable polymer tube equipped with a first ring electrode 114, a second electrode 116, and a third electrode 118. An internal coil 132 extends at least along the distal end 110 and may extend to the proximal end 170 of the pacing lead 100. The internal coil defines a lumen along the central axis thereof, which is sized to slidingly accept the elongated stylet therein. The internal lumen 132 has a distal opening 134 allowing the elongated stylet to extend forward from the distal end 110 of the pacing lead 100. A polymer sleeve 133 may surround the internal coil 132 to electrically isolate thereof from the spiral bundle 130. Spiral bundle 130 may extend from the distal end 110 to the proximal end 170 and carry individual wires connecting respective distal lead electrodes to proximal lead connectors. The example described in FIGS. 4 to 6 has four individual wires, connected respectively to the first ring electrode 114, the second coil electrode 116 via spiral wire 140, the third coil electrode 118 through the spiral wire 142, and the helical tip 120, which serves as the most distal lead electrode of the pacing lead 100.

In embodiments, the fourth spiral wire may not necessarily extend all the way from the spiral bundle 130 to the helical tip 120, but rather, it may make an electrical connection with the internal coil 132. A metal sleeve 136 may be attached to both the distal portion of the internal coil 132, as well as to the beginning of the helical tip 120 via a welded connection 138. This allows not only the retention of the helical tip 120 at the distal opening of the distal end 110, but also allows electrical communication between the helical tip 120 through the metal sleeve 136, through the internal coil 132, and to the fourth wire of the spiral bundle 130.

Exemplary dimensions of the pacing lead 100 are as follows. The pacing lead 100 may be sized to be from 0.8 mm to 2.5 mm in diameter, and in particular from 1.4 mm to 1.8 mm in diameter. The helical tip may extend from the distal end 110 by about 1.5 to 7 mm, and in particular by 2.5 to 3.0 mm. The first ring electrode 114 may be about 4-8 mm long and may extend from the distal opening at the distal end 110 by 5-15 mm. The second coil electrode 116 and the third coil electrode 118 may be 2-4 mm long and may be spaced apart from each other and from the distal end of the pacing lead by about 2-4 mm or so. Those skilled in the art may appreciate that the numbers given above are exemplary and not limiting.

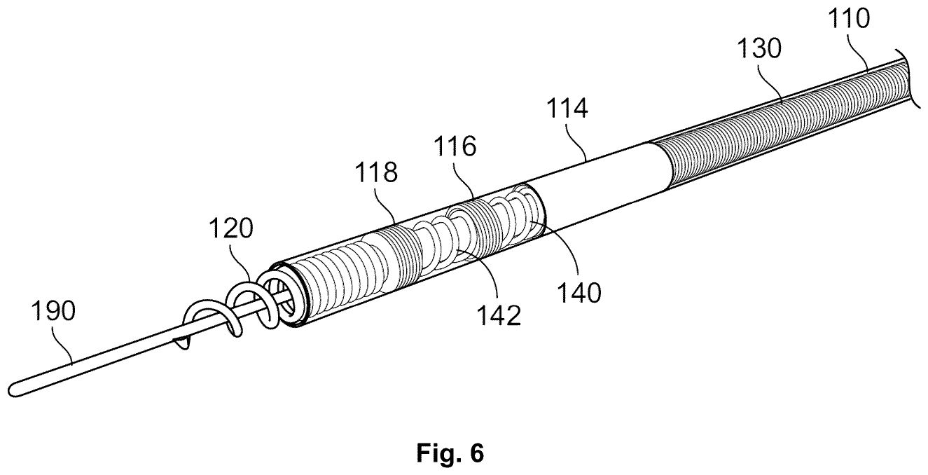

FIG. 6 illustrates the pacing lead as shown in FIGS. 4 and 5, with the addition of the elongated stylet having the stylet electrode 190 at the distal end thereof.

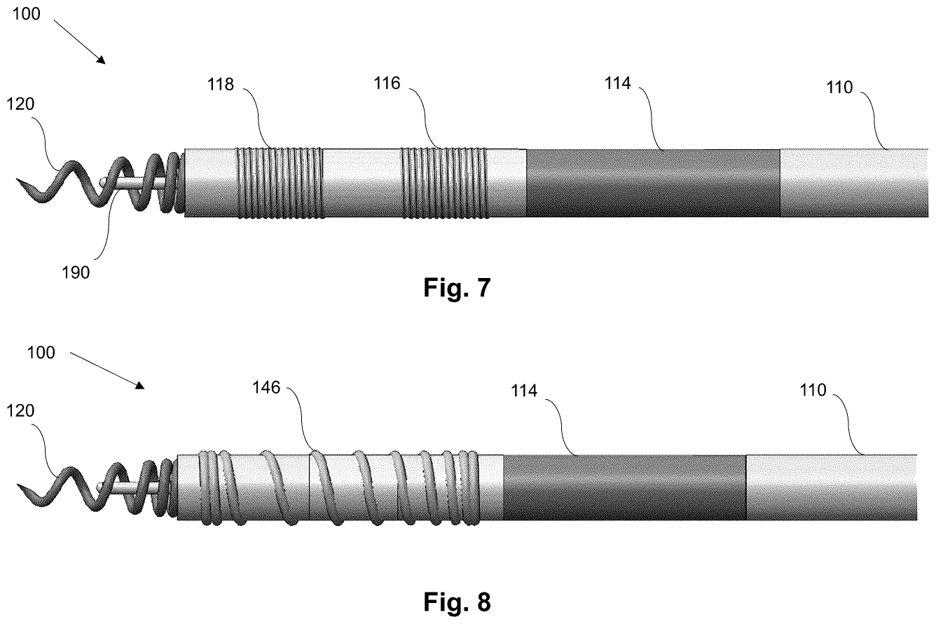

FIG. 7 shows another embodiment of the present invention. The distal end 110 of the pacing lead 100, in this embodiment, features longer second and third coil electrodes 116 and 118 as compared to the previous embodiment. The wire for the electrodes may be selected to be thinner to protrude only minimally beyond the external diameter of the pacing lead 100.

FIG. 8 shows a further embodiment of the pacing lead 100, in which the second coil electrode 146 is elongated and is stretched over most of the length of the most distal portion of the distal end 110 between the ring electrode 114 and the helical tip 120. One advantage of this arrangement is that the wire firming the second coil electrode 146 may extend beyond the outer diameter of the distal end 110 and act as a thread to additionally retain the pacing lead 100 in the cardiac tissue upon deployment thereof. Additional electrodes shown in FIG. 7 may also be used for the electrode fixation but with a lesser fixation force.

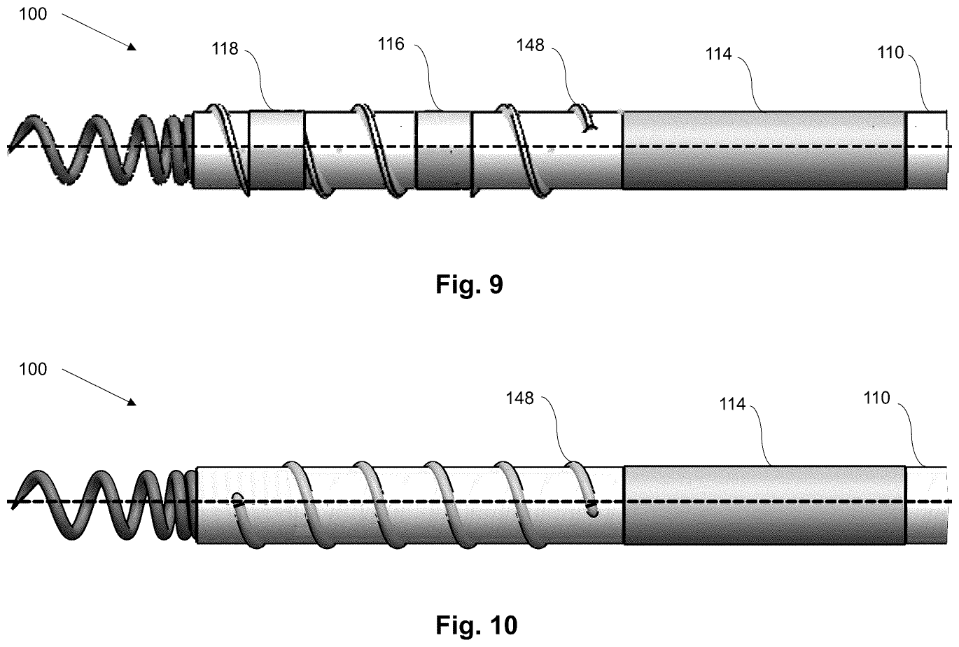

FIG. 9 shows a further embodiment of the pacing lead 100. The distal lead electrodes 114, 116, and 118 are made as ring electrodes. The additional spiral wire 148 may be made to not have any electrical connections and configured to serve only as an additional retaining/fixation element for the distal end 110.

FIG. 10 shows yet another embodiment of the present invention in which the passive external thread 148 is formed over the distal end 110 of the pacing lead 100 to aid in the retention/fixation and threading of the pacing lead 100 in the cardiac tissue. This version of the pacing lead features only two active electrodes, the helical tip 120 and the ring electrode 114. The external thread 148 may be evenly (or with a variable step) stretched over the distal end 110 of the elongated body 112 past the ring electrode 114.

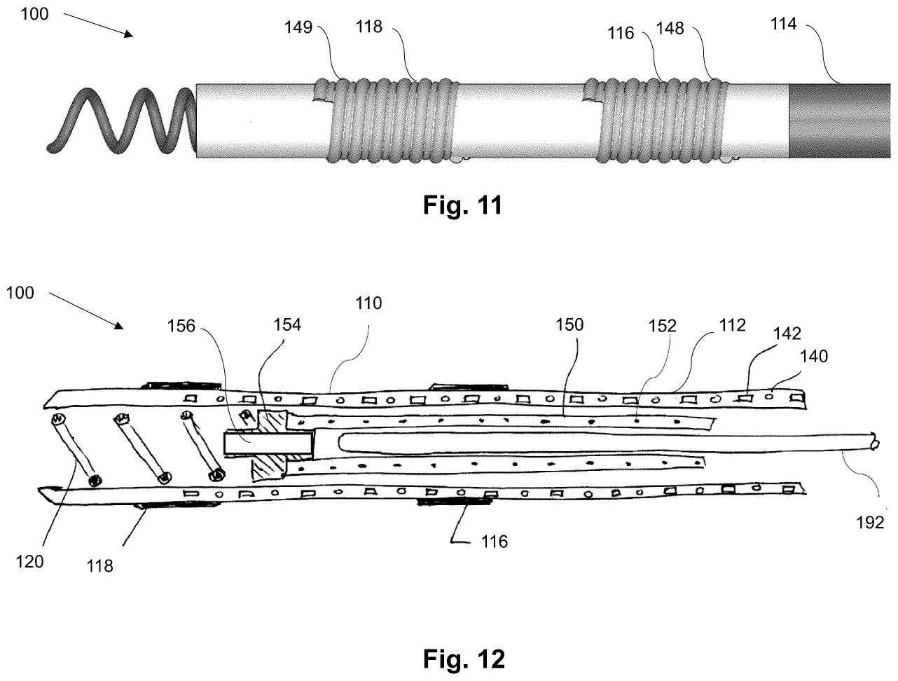

FIG. 11 shows a further yet embodiment of the present invention featuring two coil electrodes 116 and 118 made with smaller diameter wire, such as from about 0.1 mm to 0.2 mm. A larger diameter wire, such as one from 0.2 mm to 0.4 mm, runs in parallel with the coil electrode wires and may not be electrically active. It may only serve to aid in the retention/fixation of the pacing lead 100 in the cardiac tissue.

Pacing Lead with an Extendable Helical Tip

FIGS. 12 through 14 show examples of a novel pacing lead configured for use with the elongated stylet extending therethrough and featuring a helical tip extendable from the distal end 110 thereof.

FIG. 12 shows a distal end of the pacing lead 100 according to yet another embodiment of the invention. The elongated body 112 may be made from an insulating polymer material selected to assure the necessary flexibility of the pacing lead. A plurality of individual coiled wires 140 and 142 or a combined coiled bundle (not shown in the drawing) may be incorporated within the wall of the elongated body 110 to carry electrical signals between the distal end 110 and the proximal end 170 of the pacing lead 100. In embodiments, 2, 3, 4, 5, 6, 7, 8, 9, 10, 11, or more individual wires may be used to power up and carry electrical signals to a plurality of distal electrodes, a defibrillator coil, or other electrically active parts of the pacing lead 100. The wires may form a flat bundle so as not to increase the thickness of the elongated body 112. A ring electrode 118 may be operatively connected with one or more of these individual wires, for example, with the wire 140. The ring electrode 118 may be spaced away from the distal tip by about 5-13 mm, such as by 5, 6, 7, 8, 9, 10, 11, 12, 13 mm, or another suitable distance, as the invention is not limited in this regard. The length of the ring electrode may be anywhere from about 3 mm to about 10 mm, such as 3, 4, 5, 6, 7, 8, 9, 10 mm, or any other suitable length. One or more intermediate distal ring electrode 116 may, in turn, be connected to the other wires, such as wire 142 in this example. The intermediate ring electrode 116 may be spaced away from the distal tip of the lead by 3-10 mm, such as by 3, 4, 5, 6, 7, 8, 9, 10 mm, or by another suitable distance. The length of the intermediate ring electrode 116 may be selected to be 1-6 mm, such as 1, 2, 3, 4, 5, or 6 mm, as the invention is not limited in this regard. In embodiments, 1, 2, 3, 4, 5, 6, 7, 8, or more intermediate electrodes (either ring- or coil-type) may be individually connected with a respective wire to energize thereof, as the invention is not limited in this regard.

In a further alternative of the invention, the length of the intermediate ring electrode 116 may be selected to be elongated, such as 5-12 mm, for example, 5, 6, 7, 8, 9, 10, 11, 12 mm. In this case, as the intermediate electrode 116 enters the cardiac tissue, its impedance measurement will include a combination of tissue impedance and blood impedance. A shift in the overall impedance may be detected and used as a measure of the depth of penetration of the intermediate ring electrode 116 into the cardiac tissue.

An internal flexible tube 150 may be slidingly placed inside the elongated body 112 with the ability to rotate and advance inside thereof by manipulating the internal tube 150 from the proximal end of the pacing lead 100. The internal tube 150 may also be made from an insulating polymer material and may include at least one wire 152 embedded therein. A distal bushing 154 may be placed at the distal end of the internal tube 150 with the wire 152 connected thereto, which may be used to electrically connect the bushing 154 on the proximal end thereof with electrical connectors on the proximal end 170 of the pacing lead 100. The distal end of the bushing 154 may be electrically and mechanically attached (welded or soldered, for example) to the helical tip 120. Rotation and advancement of internal tube 150 may be used to urge the helical tip 120 to emerge from the distal opening of the elongated body 112 and engage with the cardiac tissue after positioning the pacing lead at the target implantation location.

The internal tube 150 may, in turn, be made hollow with an open central lumen which may be occupied by a removable elongated stylet 192. One useful purpose of including the elongated stylet 192 within the central lumen of the internal tube 150 at the beginning of the implantation procedure is to increase the stiffness of the pacing lead 100 during the insertion steps of the procedure. Other useful purposes include using the elongated stylet for temporary pacing, mapping of the heart, determination of implantation depth, stabilization of the distal end of the pacing lead, improving orthogonality of lead implantation, etc—as described elsewhere in this description.

The bushing 154 may have an opening 156 inside thereof sized to allow the elongated stylet 192 to advance through the internal tube 150 and emerge past the helical tip 120. Removal of the elongated stylet 192 at the end of the procedure may help to reduce the overall stiffness of the pacing lead and improve its conformance to the patient's anatomy. In addition, an empty channel along the central lumen of the internal tube 150 may be used to inject medication, contrast, or for other purposes during and after the initial implantation of the pacing lead 100.

In use, the pacing lead 100 of this embodiment may be positioned first at the implantation site. The helical tip 120 may be stowed inside the elongated body 112. The elongated stylet 192 may be positioned inside the internal tube 150. After the distal end is positioned at the first implantation site, the elongated stylet 192 may be advanced forward and used for pre-checking the suitability of the implantation position. Once the implantation position is identified, the helical tip 120 may be advanced to emerge from the distal end 110 and engage with the cardiac tissue. The helical tip 120 may then be advanced to the location of the stylet electrode 190. Remaining portion of the pacing lead 100 may follow the helical tip 120 until the entire pacing lead 100 is fully implanted at the confirmed position in the cardiac tissue. As an alternative, or during a part of the implantation procedure, rotation of the pacing lead 100 and the elongated stylet 192 may be implemented to avoid autorotation of the helical tip 120 back inside. Fixation of the elongated stylet 192 to the pacing lead 100 may be done at a proximal end. During the procedure, the operator may detect the implantation depth from known dimensions of the elongated body 112 and the location of the ring electrodes 116 and 118 thereon.

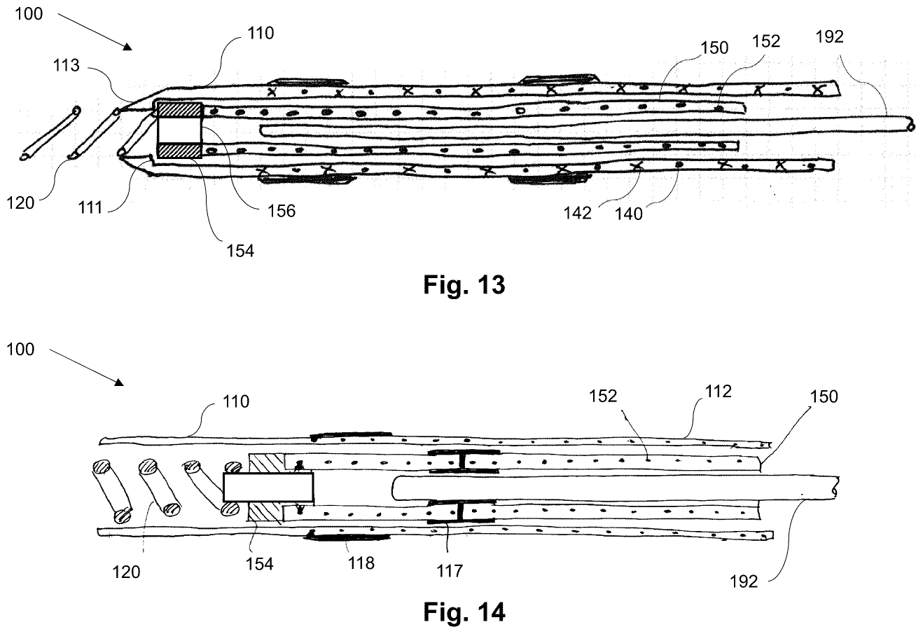

FIG. 13 shows another embodiment of the pacing lead 100 with an extendable helical tip 120, which offers an alternative design by providing an internal shoulder 111 at the distal end 110 of the elongated body 112. The bushing 154 may be made to exceed the diameter of the opening at the shoulder 111. Doing so will limit the extent of protrusion of the helical tip 120 through the distal opening of the elongated body 112, as seen in FIG. 13. Once the bushing 154 has reached the shoulder 111, further rotation of the helix tip 120 will automatically urge the entire pacing lead 100 to submerge into the cardiac tissue including the distal end 110 of the elongated body 112. Rotation of the entire elongated body 112 may be required in addition to advancing the helix tip 120 for a deeper engagement of the pacing lead 100 inside the target cardiac tissue. To ease the engagement of the distal end 110 with the cardiac tissue, a taper 113 at the distal end 110 may be provided to transition the smaller diameter of the distal end 110 opening to the larger external diameter of the elongated body 112. As with the previous embodiment, once the desired depth of implantation has been achieved, further rotation of the pacing lead 100 may be stopped, and the elongated stylet 192 may be removed.

FIG. 14 shows another yet embodiment of the pacing lead with extendable helical tip of the present invention in which the elongated stylet 192 is used to both stiffen the pacing lead during insertion and also to carry a function of a temporary electrode as described above. The elongated body 112 may be made similar to the previous embodiments of the invention. The internal tube 150 may be made to carry two or more independent coiled wires within the wall or a lumen thereof in a manner similar to that described for the previous embodiments. Intermediate ring electrode 118 (one or more if desired) may be made by a ring positioned externally to the internal tube 150 and located distally to the bushing 154 and the helical tip 120. The ring of the ring electrode 118 may be energized by connecting to one or more of the internal wires 152, which may be located within the wall of the internal tube 150. The elongated stylet 192 may be made to electrically engage with the internal ring 117, which also forms a part of the ring electrode 118. In this case, the elongated stylet 192 may be made to carry the electrical signal between the intermediate ring electrode 118 and the proximal connector of the pacing lead 100. A suitable change in the use of proximal connectors and electrical communications is needed in that case as compared to other embodiments of the invention.

As can be appreciated by those skilled in the art, additional features of the distal end 110 pf the pacing lead 100 described above for the pacing lead with a fixed helical tip and shown in FIGS. 7 to 11, may be applicable to the pacing lead with extendable helical tip as well.

### Proximal End of the Pacing Lead

In embodiments, the proximal end of the pacing lead 100 may be bifurcated into two parts: the electrical wires of the elongated body 112, as well as the internal tube 150, may be separated into a first group of wires and a second group of wires. The first group of wires may be dedicated to permanent pacing purposes and may be connected to a first proximal connector 174 adapted for use with a conventional pacemaker. The second group of wires may be attached to a separate second connector 180 located in the vicinity of the proximal portion of the pacing lead 100—see FIG. 15. The second connector 180 may be used to attach the intermediate electrodes to the impedance monitor, which may be predominantly used during the implantation procedure. Once the insertion of the pacing lead is complete (and optionally verified by X-ray or another imaging method, by electrical performance, or by both), the second connector may be permanently separated from the lead and discarded. Alternatively, it can be covered with an insulating cover to isolate it from the body and avoid open-circuit exposure to bodily fluids.

FIG. 15 shows a schematic diagram, of the proximal end of the pacing lead 100. It may be separated at the hub 172 to form two independent IS-1 connectors, one on the wire branch 174, and the other on the wire branch 180. The first branch 174 may have two IS-1 style connectors, namely a first connector 178 and a second connector 176. The second wire branch 180 may contain a third connector 184 and a fourth connector 182. These four connectors may be individually electrically connected to respective distal lead electrodes 114, 116, 118, and 120. The elongated stylet 192 may be inserted into the straight wire branch 174. The proximal end of the elongated stylet 192 may contain a fifth proximal stylet connector 161 electrically connected to the stylet electrode 190 at the distal end thereof. In further embodiments, other types of proximal electrical connectors may be used, as the invention is not limited in this regard.

FIG. 16 shows a proximal portion of the electrical connector that may be useful for the purposes of the invention. A first connector 178 with the smallest diameter may be used to retain the entire connector 174 in the transition hub 164 of the elongated stylet 192, as explained in greater detail below. Both the first connector 178 and the second connector 176 may be used to transmit electrical signals to and from distal electrodes of the pacing lead 100.

### Elongated Stylet

The following describes various designs of the proximal end of the elongated stylet 192 and designs of a transition hub 164 associated therewith.

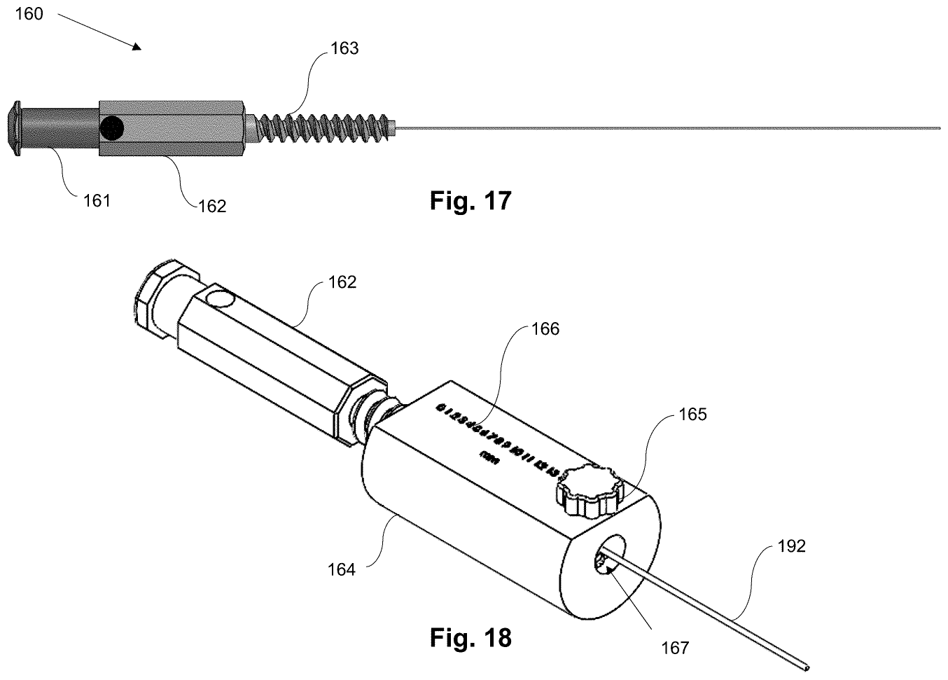

One exemplary embodiment of the proximal end of the elongated stylet 192 is seen in FIG. 17 and includes a proximal electrode connector 161, a torque handle 162, and a screw 163. The thread of the screw 163 may be selected to have one revolution per known unit of length, for example, one revolution per 1 mm of insertion depth.

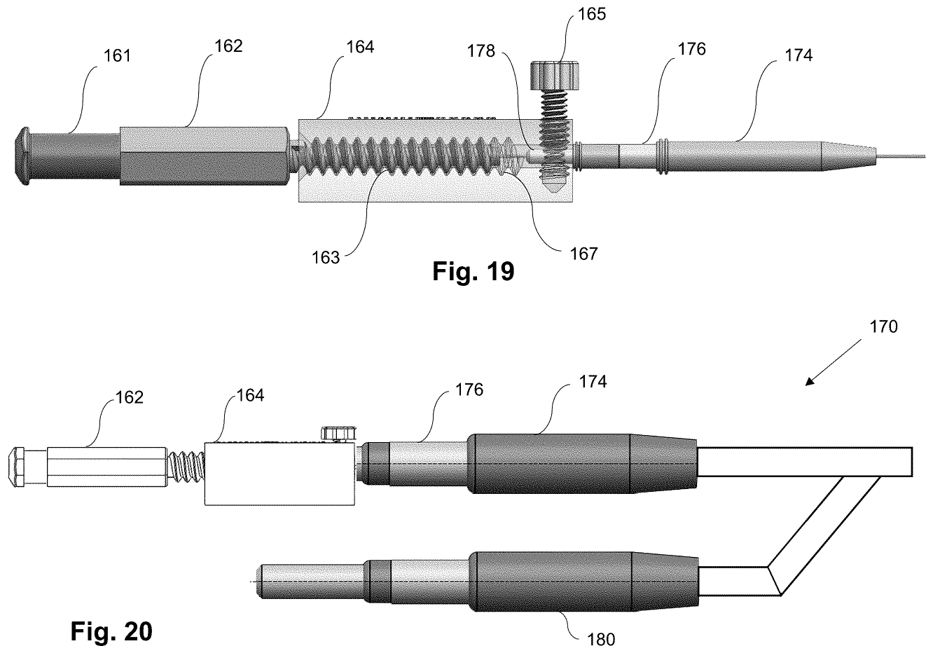

FIGS. 18 through 20 show an optionally transparent transition hub 164 having a respective threaded channel 167 inside thereof that matches the size of the screw 163. The transition hub 164 may be configured to releasably retain the elongated stylet 192 and to allow a controlled advancement of the elongated stylet in a known relationship between the position of the distal end of the elongated stylet and a distal end 110 of the pacing lead 100. In embodiments, the transition hub 164 may be made from polycarbonate. The transition hub 164 may also have a depth-indicating indicia 166 on one side thereof to indicate the depth of insertion of the elongated stylet, as explained below in greater detail. While the figures herein show a maximum insertion depth of 15 mm, the invention is not limited in this regard. The insertion depth may be selected to be anywhere from about 3 mm to about 25 mm, as limited only by the anatomical thickness of the septum. If used for a different indication or at a different location in the heart or elsewhere, the present invention may include other insertion depths as appropriate.

The transition hub 164 may have a second thread located across the threaded channel 167 and sized to accept a fixation knob 165, which allows for a fixed positioning of the transition hub 164 over the proximal electrical connector 178 of the IS-1 compatible connector 174 of the present invention.

In use, the transition hub 164 may be first positioned over the proximal electrical connector 174 while still allowing an electrical connection thereto-see FIG. 20. The transition hub 164 may be fixated in place using the knob 165. The elongated stylet 192 may then be inserted into the pacing lead of the invention from the proximal end 170, and the screw 163 is first engaged with the threaded channel 167 of the transition hub 164.

The torque handle 162 may be gradually turned as the screw 163 is slowly inserted into the transition hub 164. The end of the screw 163 is visible and may be used to monitor the degree of extension of the elongated stylet beyond the distal end of the pacing lead 100, for example, following the indicia 166 on the transition hub 164. The length of the screw 163 would limit the extent of protrusion of the stylet electrode 190 from the distal end 110 of the pacing lead 100. In other words, once the torque handle 162 reaches the body of the transition hub 164, any further insertion of the elongated stylet 192 out of the proximal end 110 is avoided. Once the pre-check of the insertion position and implantation of the pacing lead 100 is complete, the transition hub 164 may be disconnected from the proximal end 170, and the handle 162 may be removed together with the rest of the elongated stylet 192.

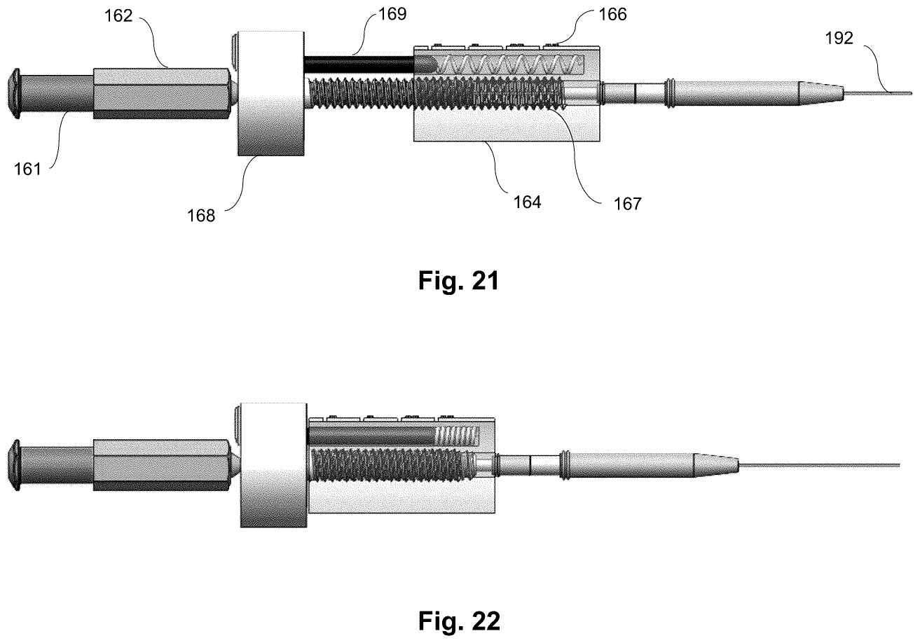

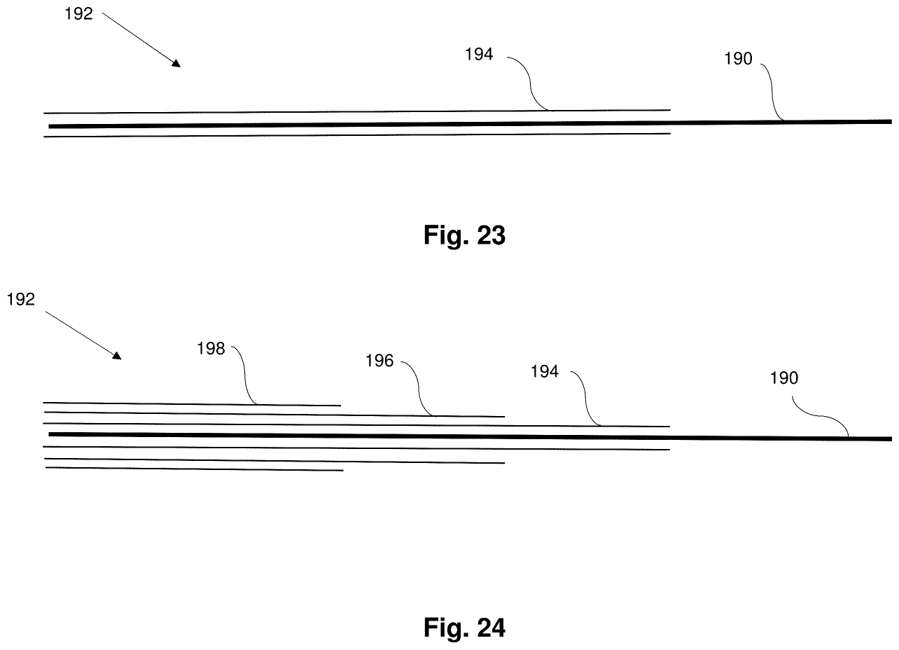

FIGS. 21 and 22 show another embodiment of the proximal end 160 and the transition hub 164. In this case, the back handle 162 serves not as the active component that is used to cause protrusion of the elongated stylet 192 from the distal end of the pacing lead 100, but as a depth-limiting component only. Individual components of this embodiment include the elongated stylet 192 with the handle 162 and the stylet electrode 161, a depth-limiting knob 168 with its dedicated screw, a spring-loaded depth indicator 169, and a transparent transition hub 164 having depth indicia 166 located on one side thereof. The depth-limiting knob 168 is threadedly inserted into a corresponding threaded channel 167 inside the transition hub 164. Turning the depth-limiting knob 168 causes its screw to advance deeper into the threaded channel 167 inside the transition hub 164, while the spring-loaded depth indicator 169 advanced inside the corresponding parallel channel inside the transition hub 164 so that the depth of protrusion is seen on the corresponding indicia 166. Once the depth-limiting knob 168 is inserted to the desired maximum depth, the actual protrusion of the elongated stylet 192 is accomplished by pushing the elongated stylet inside the pacing lead 100 using the handle 162 (the screw 163 may be omitted). Abutting the handle 162 against the depth-limiting knob 168 causes the advancement of the elongated stylet 192 to stop, thereby limiting the protrusion of thereof from the distal end 110 of the pacing lead 100 of the present invention. FIG. 21 shows a configuration suitable for a minimum depth of stylet protrusion, while FIG. 22 shows the configuration for the maximum depth of stylet insertion. The actual maximum protrusion depth may be selected by the operator depending on specific anatomical considerations for each individual patient.

While the above discussion shows a transition hub 164 as a stand-alone component, in further embodiments of the invention, the transition hub 164 may be incorporated at the proximal end of the pacing lead 100.

The following describes various distal end designs suitable for the electrically active elongated stylet of the present invention, as seen in FIGS. 23 and 24. The elongated stylet 192 of the invention may have a stylet electrode 190 at the distal end thereof, as seen in a closeup diagram in FIG. 23. The rest of the body of the elongated stylet may be optionally covered with an isolating coating or a thin sheath 194, such as a PTFE coating, in one example. The proximal end 160 of the elongated stylet 192 may be configured to be electrically connected to a cardiac stimulator and monitor (not shown) through a snap-on connection area 161, as discussed above and shown in FIGS. 17 through 22. The elongated stylet 192 may be made from stainless steel, Nitinol, or another suitable metal. The distal end of the elongated stylet 192 may be made straight or contain a fixation spiral tip (not shown) configured to temporarily deploy and retain the elongated stylet 192 in the cardiac tissue. In further embodiments, the initial shape of the distal end may be pre-formed to have a desired curve profile allowing reaching further away from the position of the distal end 110 of the pacing lead 100, as the invention is not limited in this regard.

In further yet embodiments, the elongated stylet 192 may have more than one distal stylet electrode, as illustrated in FIG. 24. In this case, additional conductive and insulating layers are provided to extend along the elongated stylet. They may be individually terminated to form or support additional stylet connectors at the proximal end of the elongated stylet. The additional stylet connectors may individually correspond to additional distal stylet electrodes at the distal end of the elongated stylet. To accomplish this, the initial electrically conductive material of the elongated stylet 192 may be first coated with an electrically isolating coating 194, exposing a conductive distal end as a stylet electrode 190. A second electrically conductive coating 196 may be applied over the non-conductive coating 194 at a predefined distance further away from the distal end of the elongated stylet 192. Finally, a third non-conductive coating 198 may be applied to the external surface of the elongated stylet 192 with a further step back from the end of the conductive coating 196, thereby forming a second distal stylet electrode in the area where the second conductive coating 194 has an exposed outer surface to be in contact with blood or cardiac tissue. A similar arrangement may be applied to the proximal end 160 of the elongated stylet 192 so as to create suitable contact zones and/or proximal stylet connectors for facilitating activation of the stylet electrode 190 and additional distal stylet electrodes at the distal end of the elongated stylet 192 as appropriate.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An elongated stylet comprising at least one stylet electrode located at a distal end thereof and configured to be electrically connected to a stylet connector at a proximal end thereof, which, in turn, is configured to transmit electrical signals to or from the stylet electrode, the elongated stylet is configured to slidingly fit in and protrude from a lumen of a cardiac pacing lead.

2. The elongated stylet, as in claim 1, further comprising an electrically insulated external layer extending along thereof between the stylet connector and the stylet electrode.

3. The elongated stylet, as in claim 1, further comprising additional conductive and insulating layers extending along thereof and terminated progressively further away from the distal end thereof to form additional spaced apart distal stylet electrodes.

4. The elongated stylet, as in claim 3, wherein additional conductive and insulating layers extending along the elongated stylet are individually terminated to form additional stylet connectors at the proximal end of the elongated stylet, the additional stylet connectors individually correspond to additional distal stylet electrodes at the distal end of the elongated stylet.

5. A cardiac pacing lead comprising an elongated flexible body with at least one lead electrode positioned at a distal end thereof, the lead electrode is electrically connected to a lead connector at a proximal end of the elongated body, which, in turn, is configured to transmit electrical signals to or from the distal lead electrode, wherein the elongated body comprising a lumen sized to slidingly accept an elongated stylet positioned therein,
wherein the elongated stylet comprising at least one stylet electrode located at a distal end thereof and configured to be electrically connected to a stylet connector at a proximal end thereof, which, in turn, is configured to transmit electrical signals to or from the stylet electrode.

6. The cardiac pacing lead, as in claim 5, further comprising a transition hub configured to releasably retain the elongated stylet therein and to allow a controlled advancement of the elongated stylet in a known relationship between the position of the distal end of the elongated stylet and the distal end of the pacing lead.

7. The cardiac pacing lead, as in claim 6, wherein the transition hub is made from a transparent material and contains indicia indicating insertion depth of the elongated stylet in a known relationship to the distal end of the pacing lead.

8. The cardiac pacing lead, as in claim 7, wherein the transition hub comprises a threaded channel configured to retain and facilitate advancement of the elongated stylet through the lumen of the pacing lead.

9. The cardiac pacing lead, as in claim 8, wherein the threaded channel of the transition hub is configured to accept a corresponding torque handle at the proximal end of the elongated stylet to facilitate a metered advancement of the elongated stylet inside the lumen of the pacing lead by rotating the torque handle of the elongated stylet, while the torque handle of the elongated stylet cooperates with the threaded channel of the transition hub.

10. The cardiac pacing lead, as in claim 9, wherein the transition hub is configured to removably attach to the proximal end of the pacing lead.

11. The cardiac pacing lead, as in claim 9, wherein the transition hub is incorporated at the proximal end of the pacing lead.

12. A method of implanting a cardiac pacing lead for providing a pacing therapy to a target cardiac tissue comprising the steps of:
a. providing the cardiac pacing lead with a lead electrode at a distal end thereof and a lumen sized to slidingly accept an elongated stylet positioned therein, wherein the elongated stylet comprising at least one stylet electrode located at a distal end thereof and configured to be electrically connected to a stylet connector at a proximal end thereof, which, in turn, is configured to transmit electrical signals to or from the stylet electrode,
b. positioning a distal end of the pacing lead adjacent to a target cardiac tissue,
c. advancing the elongated stylet through the lumen in the cardiac pacing lead to position the stylet electrode at a first position for implantation of the cardiac pacing lead,
d. pre-check the first position for implantation in the cardiac tissue by temporarily confirming the ability to deliver the pacing therapy thereat using the stylet electrode,
e. if the confirmation is not reached in step (d), changing the depth of the stylet electrode of the elongated stylet or repositioning the distal end of the cardiac pacing lead and the elongated stylet to a second or further position and repeating step (d) of temporarily confirming the ability to deliver the pacing therapy at the current position of the stylet electrode,
f. upon reaching the confirmation in step (d) or step (e), deploying the cardiac pacing lead to locate the lead electrode next to the stylet electrode of the elongated stylet, and
g. withdrawing the elongated stylet while leaving the cardiac pacing lead in the target cardiac tissue.

13. The method of implanting a cardiac pacing lead, as in claim 12, wherein the step of temporarily confirming the ability to deliver the pacing therapy using the elongated stylet further comprises a step of pacing using the stylet electrode or a step of monitoring an intracardiac electrogram signal acquired using the stylet electrode at the distal end of the elongated stylet.

14. The method of implanting a cardiac pacing lead, as in claim 12, wherein the step of pre-checking the position for implantation in the cardiac tissue by temporarily confirming the ability to deliver the pacing therapy thereat comprises a step of confirming the pacing location using a predetermined acceptability criterion.

15. The method of implanting a cardiac pacing lead, as in claim 12, wherein in step (a), the distal end of the elongated stylet further comprises at least one additional distal stylet electrode, and wherein in steps (d) or (e) the step of temporarily confirming the ability to deliver the pacing therapy further comprises a step of temporary pacing or monitoring an intracardiac electrogram using any of the stylet electrode or additional distal stylet electrodes in a unipolar fashion or in a bipolar fashion.

16. The method of implanting a cardiac pacing lead, as in claim 12, further comprising a step of improving orthogonality of lead implantation after step (b) and before step (c), wherein the pacing lead is manipulated to improve orthogonality once the distal end thereof is provisionally engaged with the target cardiac tissue.

17. The method of implanting a cardiac pacing lead, as in claim 12, further comprising a step of using any available electrodes of the elongated stylet or the pacing lead for monitoring impedance to determine implantation depth of the respective elongated stylet or the pacing lead.

18. The method of implanting a cardiac pacing lead, as in claim 12, wherein the pacing therapy is selected to pace a left bundle branch area, and wherein in step (d) or (e) the step of temporarily confirming the ability to deliver the pacing therapy further comprises a step of temporary pacing the left bundle branch area using the stylet electrode of the elongated stylet to verify the ability to capture thereof.
